(19) United States
(12) Patent Application Publication
(10) Pub. No.: US 2024/0003863 A1
(43) Pub. Date: Jan. 4, 2024

(54) METHODS FOR MEASURING ORGANIC CARBON CONTENT OR AGRICULTURAL SOILS

(71) Applicant: Nutrien AG Solutions, Inc., Loveland, CO (US)

(72) Inventor: Matthew M. Duncan, Loveland, CO (US)

(21) Appl. No.: 18/213,074

(22) Filed: Jun. 22, 2023

Related U.S. Application Data
(60) Provisional application No. 63/357,481, filed on Jun. 30, 2022.

(51) Int. Cl. G01N 33/24; A01B 79/00

(52) U.S. Cl. CPC G01N 33/24; A01B 79/005; G01N 2033/245

(57) ABSTRACT

Soil carbon measurement methods described herein provide enhanced accuracy and precision. One example is a method for determining organic carbon content in agricultural field soil used to grow crops. Embodiments include measuring bulk density of the soil at a sample site of the agricultural field in situ to obtain bulk density data, measuring percentage of carbon in the sample site soil of the agricultural field to obtain percentage of carbon data, and determining the organic carbon content of the soil based on the bulk density data and the percentage of carbon data. Measuring the bulk density of the soil may include performing a nuclear density measurement at the sample site, such as for example by operating a nuclear density gauge including a radioactive source exempt from licensing by applicable governmental laws or regulations.

### REFERENCE TO RELATED APPLICATION

This application claims the benefit of U.S. Provisional Application No. 63/357,481, filed Jun. 30, 2022 and entitled Methods for Measuring Organic Carbon Content of Agricultural Soils, which is incorporated herein by reference in its entirety and for all purposes.

### FIELD

This disclosure relates generally to methods for determining amounts of organic carbon in agricultural soils.

### BACKGROUND

Carbon sequestration refers to processes that include capturing and storing carbon dioxide (CO₂) and other forms of carbon in a reservoir. Incentives for carbon sequestration include its ability to mitigate climate change. For example, economic incentives may be provided to growers to use agricultural practices that produce carbon sequestration.

For these and other reasons it is important to be able to accurately measure or determine the amounts of carbon present in agricultural soils. Accurate measurements of soil carbon quantities facilitates accurate quantification of changes in carbon quantities over time.

Known approaches for measuring the amounts of carbon in soil include collecting samples of the soil of interest (e.g., in an agricultural field), and processing the soil samples. These measurement approaches may make use of the bulk density of the soil, such as for example grams/cubic centimeter (g/cm³). Accordingly, known volumetric quantities of the soil are collected during the sampling process. Soil sample probes in the form of cylindrical tubes that are inserted into the soil to predetermined depths and withdrawn with a predetermined sample volume are often used for this purpose. The known volumetric sample may then be dried and weighed, and whereby the density of the sample is determined.

A sample of the soil is also processed and analyzed to determine the percentage of carbon in the sample with respect to the total mass of the sample. Known approaches for determining the percentage of carbon in the sample include spectroscopy and wet and dry combustion techniques. The percentage carbon analysis may be performed using soil from the sample that was used to make the bulk density measurement, or from another sample (e.g., collected in the same area as the sample used for the density measurement). The mass of carbon per unit of area can be computed based on the results of the bulk density measurement and percentage of carbon measurement. For example, mass of carbon per unit area can be determined by Eq. 1 below.

M/A=(BD)(SOC%/100)d Eq. 1
where: D=density,
M=mass,
V=volume,
A=area,
d=depth,
BD=bulk density,
SOC %=% of soil organic carbon,
D=M/V,
D=M/Ad.

Carbon measurements such as those described above are commonly determined on the basis of relatively small soil samples. The results of these measurements may be characterized in relatively small units such as g/cm³ corresponding to the sample sizes. However, matters relating to carbon sequestration in connection with agricultural practices may be based on larger scales, such as for example tonnes of carbon per acre. The total amounts of carbon at the scale of interest is extrapolated from the units of the measurement. For some approaches, the amounts of carbon are determined for areas having depths of about 12 inches or 30 cm. For example, carbon measurements in units of g/cm³ can be scaled up to units of tonnes/acre by multiplying the measurement by an appropriate factor such as 1.35. Because of this approach of using relatively small samples to determine the amounts of carbon, relatively small amounts of variability or uncertainty in the measurements may result in relatively large variability or uncertainty at the size scales used in agricultural applications.

There is, therefore, a need for improved methods for measuring or determining carbon content in soil. Methods that facilitate enhanced accuracy and/or precision of the determinations, for example by reducing variability or uncertainty, especially at larger scales such as tonnes/acre as used in agricultural applications, may enhance confidence in the measurements, and encourage adoption and acceptance of economic incentives for carbon sequestration practices in agricultural applications. Such methods that may be relatively easily and efficiently performed would be especially desirable.

### SUMMARY

Soil carbon measurement methods described herein provide enhanced accuracy and precision. Variability and uncertainty associated with prior art approaches may be reduced. The methods can also be relatively easily and efficiently performed. Increased numbers of soil samples and associated measurements may thereby be efficiently taken over a given area of land and used to determine the quantity of carbon in that area, further enhancing the accuracy or precision of the carbon content determination for the area.

A first example includes a method for determining organic carbon content in agricultural field soil used to grow crops, and may comprise: measuring bulk density of the soil at a sample site of the agricultural field in situ to obtain bulk density data; measuring percentage of carbon in the sample site soil of the agricultural field to obtain percentage of carbon data; and determining the organic carbon content of the soil based on the bulk density data and the percentage of carbon data. In some embodiments, measuring the bulk density of the soil includes performing a nuclear density measurement at the sample site. In any or all embodiments of this example, performing the nuclear density measurement includes operating a nuclear density gauge including a radioactive source. In any or all embodiments of this example, operating the nuclear density gauge includes operating a nuclear density gauge exempt from licensing by applicable governmental laws or regulations.

In any or all embodiments of this example, measuring the bulk density of the soil includes measuring the bulk density of soil undisturbed for the measurement.

In any or all embodiments of this example, measuring the bulk density of the soil includes operating a density measurement instrument at the sample site. For example, operating the density measurement instrument at the sample site may include: inserting a probe into the soil at the sample site; and receiving a visual display of the bulk density data from the instrument at the sample site.

Any or all embodiments of this example may further comprise collecting a soil sample of the sample site soil of the agricultural field; and measuring percentage of carbon in the sample site soil may comprise measuring the percentage of carbon in the soil sample. For example, measuring the bulk density of the soil includes obtaining the bulk density data at the sample site; and the method may further comprise: transporting the soil sample to a location remote from the sample site; measuring the percentage of carbon in the sample site soil includes measuring the percentage of carbon in the soil sample at the location remote from the sample site; and determining the organic carbon content of the soil includes determining the organic carbon content at a location remote from the sample site. In any or all of these embodiments, determining the organic carbon content of the soil includes determining the organic carbon content of the soil at the location that the percentage of carbon in the soil was measured.

In any or all embodiments of this example, determining the organic carbon content includes: determining the organic carbon content in the soil sample in a first set of units referenced to a first volume; and multiplying the organic carbon content in the first set of units by a scaling factor to determine the organic carbon content in a second set of units referenced to a second volume greater than the first volume. For example, multiplying by a scaling factor includes multiplying by a scaling factor to determine the organic carbon content in a second set of units suitable for an agricultural application. For example, the second set of units comprises tons of carbon per acre.

In any or all embodiments of this example, the steps of the method may be performed for at least five sample sites per acre of a field including a plurality of contiguous acres to determine the organic carbon content at each of the at least five sample sites per acre; and include determining organic carbon content for the field based on the organic carbon content determined at the at least five sample sites per acre.

In any or all embodiments of this example, the method further comprises measuring moisture content of the sample site soil to obtain moisture data; and determining the organic carbon content further comprises determining the organic carbon content of the soil based on the moisture data. For example, measuring the moisture content may comprise measuring the moisture content of the sample site soil in situ. For example, measuring the bulk density may comprises measuring the bulk density of the sample site soil with a first instrument; and measuring the moisture content comprises measuring the moisture content of the sample site soil with a second instrument different than the first instrument.

In any or all embodiments of this example, the method may be used for determining the organic carbon content in agricultural field soil used to grow annual crops. For example, the method may be used for determining the organic carbon content in agricultural field soil used to grow row crops.

A second example includes a method for determining organic carbon content in agricultural field soil used to grow crops. Embodiments may comprise determining the organic carbon content in the agricultural field soil in accordance with the method of any or all embodiments of the first example before planting or substantial growth of the crops; determining the organic carbon content in the agricultural field soil in accordance with the method of any or all embodiments of the first example after substantial growth or harvest of the crops; and determining carbon sequestration in the agricultural field soil based on the organic carbon content before planting or substantial growth of the crops and the organic carbon content after substantial growth or harvest of the crops.

A third example includes a method for determining organic carbon content in agricultural field soil used to grow crops. Embodiments may comprise: receiving in situ-measured bulk density data of the soil of the agricultural field at a sample site, wherein the bulk density data was determined by measuring the bulk density of the sample site soil at the sample site in situ; receiving a soil sample of the sample site soil of the agricultural field, wherein the soil sample was collected at the sample site; measuring percentage of carbon in the soil sample to obtain percentage of carbon data; and determining the organic carbon content of the soil based on the bulk density data and the percentage of carbon data. The bulk density data was measured by performing a nuclear density measurement at the sample site in embodiments. The bulk density data was measured by operating a nuclear density gauge including a radioactive source in embodiments. The bulk density data was measured by operating a nuclear density gauge exempt from licensing by applicable governmental laws or regulations in embodiments.

In any or all embodiments of this example, the bulk density data was measured in sample site soil undisturbed for the measurement.

In any or all embodiments of this example, the bulk density data was measured by operating a density measurement instrument at the sample site. In any or all embodiments of this example, the density measurement instrument was operated by: inserting a probe into the soil at the sample site; and receiving a visual display of the bulk density data from the instrument at the sample site.

In any or all embodiments of this example, the bulk density data was obtained at the sample site; the soil sample was transported to a location remote from the sample site; measuring the percentage of carbon in the soil sample includes measuring the percentage of carbon in the soil sample at the location remote from the sample site; and determining the organic carbon content of the soil includes determining the organic carbon content at a location remote from the sample site. For example, determining the organic carbon content of the soil may include determining the organic carbon content of the soil at the location that the percentage of carbon in the soil was measured.

In any or all embodiments of this example, determining the organic carbon content may include: determining the organic carbon content in the soil in a first set of units referenced to a first volume; and multiplying the organic carbon content in the first set of units by a scaling factor to determine the organic carbon content in a second set of units referenced to a second volume greater than the first volume. For example, multiplying by a scaling factor may include multiplying by a scaling factor to determine the organic carbon content in a second set of units suitable for an agricultural application. For example, the second set of units may comprise tons of carbon per acre.

Any or all embodiments of this example may comprise performing the steps for at least five sample sites per acre of a field including a plurality of contiguous acres to determine the organic carbon content at each of the at least five sample sites per acre; and determining organic carbon content for the field based on the organic carbon content determined at the at least five sample sites per acre.

In any or all embodiments of this example, the method further comprises receiving moisture data representative of moisture content of the sample site soil; and determining the organic carbon content further comprises determining the organic carbon content of the soil based on the moisture data. For example, the moisture data may have been obtained by measuring the moisture content of the sample site soil at the sample site in situ. For example, the bulk density data may have been obtained by measuring the bulk density of the sample site soil with a first instrument; and the moisture data may have been obtained by measuring the moisture content of the sample site soil with a second instrument different than the first instrument.

In any or all embodiments of this example, the method is used for determining the organic carbon content in agricultural field soil used to grow annual crops. For example, the method may be used for determining the organic carbon content in agricultural field soil used to grow row crops.

As a fourth example, a method for determining organic carbon content in agricultural field soil used to grow crops may comprise: determining the organic carbon content in the agricultural field soil in accordance with the method of any embodiments of the third example before planting or substantial growth of the crops; determining the organic carbon content in the agricultural field soil in accordance with the method of any embodiments of the third example after substantial growth or harvest of the crops; and determining carbon sequestration in the agricultural field soil based on the organic carbon content before planting or substantial growth of the crops and the organic carbon content after substantial growth or harvest of the crops.

### BRIEF DESCRIPTION OF THE DRAWINGS

FIG. 1 is a diagrammatic illustration of a method for determining organic carbon content in agricultural fields in accordance with embodiments.

### DETAILED DESCRIPTION

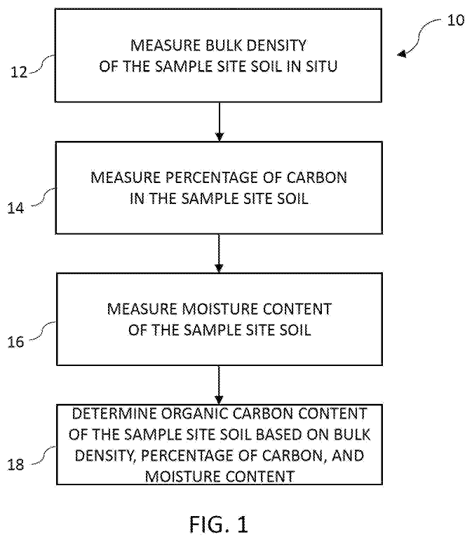

FIG. 1 is a diagrammatic illustration of a method 10 for determining organic carbon content in the soil of agricultural fields used for growing crops, in accordance with embodiments. For example, the method may be used in connection with fields growing row or other types of crops for human and/or animal consumption. The method is performed by analyzing the soil of the field. The analyzed soil may be located at and/or collected from one or more sample sites in the agricultural field. As described in greater detail below, at least portions of the analysis may be performed on the soil at the sample site. In certain embodiments, portions of the analysis may be performed at locations remote from the sample site using soil samples collected at the sample site and transported to the remote locations. As shown generally in FIG. 1, the method 10 includes (1) measuring or otherwise determining the bulk density of the sample site soil in situ (step 12), (2) measuring or otherwise determining the percentage of carbon in the sample site soil (step 14), (3) measuring or otherwise determining the moisture content of the sample site soil (step 16), and (4) computing or otherwise determining the organic carbon content of the sample site soil based upon (i) the bulk density, (ii) the percentage of carbon, and (iii) the moisture content (step 18). Conventional or otherwise known relationships, such as for example the relationship described by Eq. 1 above, can be used to compute or otherwise determine the organic carbon content from the determined quantities of bulk density (BD), and percentage of carbon (SOC %). Moisture content may also be accommodated by some approaches.

Step 12, the measurement of the bulk density of the soil at the sample site, is performed in situ. For example, the bulk density measurement of step 12 may be performed on the soil while the soil is in its natural or original place in the field. The sampled soil need not be removed from its place in the field in connection with the bulk density measurement. In embodiments, the bulk density measurement of step 12 is performed on the soil at the sample site without disturbing the soil. For example, a sample of the soil need not be physically collected (e.g., need not be removed from its then-existing location on the ground) or moved for purposes of the bulk density measurement of step 12.

In embodiments, the bulk density measurement of step 12 can be performed using a density measurement instrument operated at the sample site. An example of a density measurement instrument that can be operated to obtain soil density measurements in accordance with step 12 is the nuclear EGauge Model 4590 instrument available from Troxler Electronic Laboratories, Inc. of Research Triangle Park, North Carolina. Nuclear density measurement instruments such as the Troxler EGauge Model 4590 include a radioactive probe, detector, processing system and user interface. The probe is inserted into the soil (e.g., a hole formed in the soil) at the sample site, and emits radiation into the adjacent mass of soil. The detector detects the radiation after it passes through the mass of soil. The processing system generates information representative of the density of the soil at the sample site based on the detected radiation (e.g., radiation reflected back to the instrument). The density measurement may be visually displayed (e.g., by the user interface), stored for later retrieval or transmission, or transmitted (e.g., wirelessly) to another device (e.g., for storage or display). A particular advantage of the Troxler EGauge Model 4590 instrument is that it is exempt from certain governmental laws or regulations such as those relating to nuclear regulatory licensing in the United States, thereby enhancing the convenience of its use. Instruments such as the Troxler EGauge Model 4590 may be operated in accordance with conventional or otherwise known approaches, including those described in documents and other information sources published by the manufacturers or distributors of the instruments, to make bulk density measurements in accordance with step 12. Other embodiments of method 10 use other conventional or otherwise known nuclear or non-nuclear instruments to measure the bulk density of the soil at the sample site in situ.

Use of instruments such as the Troxler EGauge Model 4590 involves the formation of a hole in the soil at the sample site to receive the probe. However, the sample site need not be otherwise disturbed in connection with the measurement. The mass of the soil through which the radiation emitted by the probe passes in connection with the measurement (e.g., the soil for which the bulk density is being measured) is undisturbed by the density measurement procedure of step 12. During testing and development of bulk density measurement steps such as those described herein using a Troxler EGauge Model 4590 instrument, the probes were inserted into the topsoil at the sample sites to depths of about twelve inches (30 cm). As described below, highly accurate and precise bulk density measurements of the sample site soil at these depths can be obtained using the instruments and methods described herein. The bulk density measurements are also convenient and efficient to perform.

Step 14, the measurement of the percentage of carbon in the sample site soil, may be performed by conventional or otherwise known methods. Non-limiting examples of such methods include dry combustion, wet combustion and spectroscopy approaches. Conventional or otherwise known laboratory equipment and associated facilities can be used in connection with these methods.

The percentage of carbon measurements of step 14 may be performed on soil samples collected at the sample site. For example, soil samples collected at the sample sites may be transported to facilities remote from the sample sites that have the laboratory equipment suitable for performing the percentage of carbon measurements. Alternatively, portable laboratory equipment may be transported to the sample sites, or to locations near the sample sites, enabling the percentage of carbon measurements to be made at or near the sample sites. In yet other embodiments, the percentage of carbon measurements may be performed in situ.

Step 16, the measurement of the moisture content of the sample site soil, may be performed by conventional or otherwise known methods. In embodiments, the moisture content measurements of step 16 may be performed on soil samples collected at the sample site. The soil samples collected for the percentage of carbon measurements of step 14, or portions of such soil samples, may for example be used for the moisture content measurements at step 16. In embodiments, the soil samples collected at the sample sites may be transported to facilities remote from the sample sites that have the laboratory equipment suitable for performing the moisture content measurements. Alternatively, portable laboratory equipment may be transported to the sample sites, or to locations near the sample sites, enabling the moisture content measurements to be made at or near the sample sites. The moisture content measurements of step 16 may, for example, be performed at the same locations as the percentage of carbon measurements of step 14.

In yet other embodiments, the moisture content measurements of step 16 are performed in situ. For example, the moisture content measurement of step 16 may be performed on the soil while the soil is in its natural or original place in the field. The sampled soil need not be removed from its place in the field in connection with the moisture content measurement. In embodiments, the moisture content measurement of step 16 is performed on the soil at the sample site without disturbing the soil. For example, a sample of the soil need not be physically collected (e.g., need not be removed from its then-existing location on the ground) or moved for purposes of the moisture content measurement of step 16.

In embodiments, the moisture content measurement of step 16 can be performed using a moisture content measurement instrument operated at the sample site. An example of a moisture content measurement instrument that can be operated to obtain soil moisture content measurements in situ in accordance with step 16 is the Model 6760 moisture probe provided with the EGauge Model 4590 instrument described above, available from Troxler Electronic Laboratories, Inc. of Research Triangle Park, North Carolina. The Model 6760 moisture probe uses electromagnetic technology and interfaces with the EGauge Model 4590 instrument to provide moisture content measurements. The probe is inserted into the soil (e.g., a hole formed in the soil, which may be the same hole used for the bulk density measurement) at the sample site, and emits electromagnetic radiation into the adjacent mass of soil. The electromagnetic radiation is detected after it passes through the mass of soil. Information representative of the moisture content of the soil at the sample site is generated based on the detected radiation. The moisture content measurement may be visually displayed, stored for later retrieval or transmission, or transmitted (e.g., wirelessly) to another device for storage or display (e.g., by the user interface on the EGauge Model 4590 instrument). Instruments such as the Troxler Model 6760 may be operated in accordance with conventional or otherwise known approaches, including those described in documents and other information sources published by the manufacturers or distributors of the instruments, to make moisture content measurements in accordance with step 16. Other embodiments of method 10 use other conventional or otherwise known instruments to measure the moisture content of the soil at the sample site in situ.

Use of instruments such as the Troxler Model 6760 moisture probe involve the formation of a hole in the soil at the sample site to receive the instrument. However, the sample site need not be otherwise disturbed in connection with the measurement. The mass of the soil through which the electromagnetic radiation emitted by the instrument passes in connection with the measurement (e.g., the soil for which the moisture content is being measured) is undisturbed by the moisture content measurement procedure of step 16 during the use of instruments of these types. Highly accurate moisture content measurements of the sample site soil can be obtained using the instruments and methods described herein. The moisture content measurements are also convenient and efficient to perform.

Step 18, the determination of the organic carbon content in the sample site soil, can be performed based on the information produced by the associated bulk density measurements, percentage of carbon measurements and moisture content measurements described above in connection with steps 12, 14 and 16. Conventional or otherwise known algorithms such as those represented by Equation 1 above can be used for the organic carbon content determination of step 18. In embodiments, the information representative of the associated bulk density, percentage of carbon and moisture content measurements may be stored electronically in data storage. Conventional computing systems may be used to compute the organic carbon content based on the stored bulk density, percentage of carbon and moisture content measurements.

Organic carbon content determinations by step 18 may be referenced to a first volume unit, such as g/cm³ (grams per cubic centimeter). The first volume unit, e.g., cubic centimeters, is a volume based on or consistent with the volume of the sample site soil used for the measurements at one or more of steps 12, 14 or 16. Agricultural field applications for organic carbon content measurement, however, make use of the carbon content in field-level soil volume units that may be greater or substantially greater than the volume levels of the soil samples. In connection with agricultural field applications, for example, carbon contents are commonly referenced to a second and greater volume such as tonnes/acre (metric tons per acre). To determine the carbon content for the relatively larger field-level applications, the organic carbon content determinations at the relatively smaller volume levels may be multiplied by a scaling factor. For example, Eq. 2 below defines the scaling factor for converting organic carbon determination in grams per cubic centimeter to equivalent determinations in metric tons per acre.

carbon (tonnes/acre)=carbon (g/cm³)×d Eq. 2
where d=depth of soil volume of interest.

Tests of organic carbon content measurement methods such as those described above have demonstrated improved accuracy and precision over prior art methods using collected physical core samples as the bases for the bulk density measurements. By one comparative test procedure, organic carbon determinations were performed on soil at the same sample sites by a first, prior art method, and a second method in accordance with the methods described herein. By the first method, a physical soil sample collected using a cylindrical tube as described above in the Background section was analyzed using conventional processes to determine the bulk density of the sample site soil. By the second method, a Troxler EGauge Model 4590 was operated in accordance with the manufacturer's specifications to determine the bulk density of the sample site soil in situ. For both the first and second methods, the same and conventional processes were used to determine the percentage of carbon and moisture content in the soil samples.

By these tests, the second method in accordance with this disclosure demonstrated significant improvements in accuracy and precision of the organic carbon content measurements over the first, prior art method. The enhanced measurement accuracies and precision are particularly significant since they are scaled to the larger metric tonnes per acre units used for agricultural field applications.

At least in part because of the efficiency and convenience of use of methods such as 10 for determining organic carbon content, the method may be effectively performed at increased numbers of sample sites within a given agricultural field (e.g., at a greater density of sample sites), thereby increasing the granularity of the measurements, and the overall accuracy of the organic carbon content determinations in a field. In embodiments, for example, the method 10 may be performed for at least five, at least 10, or at least 15 sample sites per acre of a field including a plurality of contiguous acres to determine the organic carbon content at each of the sample sites. The organic carbon content of the field may then be based on the measured values of the organic carbon content at the multiple sample sites per acre. Agricultural fields such as these that are formed by a plurality of contiguous acres and used to grow crops, such as annual and/or row crops, may include features or structures such as roads, tree lines, fence lines and drainage streams.

Accurate and precise analyses of carbon sequestration in agricultural fields can be obtained by use of the methods such as those described herein. For example, the organic carbon content measurement methods may be performed before planting or substantial growth of the crops, and again after substantial growth or harvest of the crops. Accurate and precise assessments of carbon sequestration over the period of time between the measurements can be made based on the measurements. In other embodiments, sequestration may be based on measurements made over other periodic time frames (e.g., annually, or over multiple (e.g., 5, 10, 20) years).

It is to be understood that the above description is intended to be illustrative, and not restrictive. Many other embodiments will be apparent to those of skill in the art upon reading and understanding the above description. It is contemplated that features described in association with one embodiment are optionally employed in addition or as an alternative to features described in or associated with another embodiment. The scope of the invention should, therefore, be determined with reference to the appended claims, along with the full scope of equivalents to which such claims are entitled.

What is claimed is:

1. A method for determining organic carbon content in agricultural field soil used to grow crops, comprising:
measuring bulk density of the soil at a sample site of the agricultural field in situ to obtain bulk density data;
measuring percentage of carbon in the sample site soil of the agricultural field to obtain percentage of carbon data; and
determining the organic carbon content of the soil based on the bulk density data and the percentage of carbon data.

2. The method of claim 1, wherein measuring the bulk density of the soil includes performing a nuclear density measurement at the sample site.

3. The method of claim 2, wherein performing the nuclear density measurement includes operating a nuclear density gauge including a radioactive source.

4. The method of claim 3, wherein operating the nuclear density gauge includes operating a nuclear density gauge exempt from licensing by applicable governmental laws or regulations.

5. The method of claim 1, wherein measuring the bulk density of the soil includes measuring the bulk density of soil undisturbed for the measurement.

6. The method of claim 1, wherein measuring the bulk density of the soil includes operating a density measurement instrument at the sample site.

7. The method of claim 6, wherein operating the density measurement instrument at the sample site includes:
inserting a probe into the soil at the sample site; and
receiving a visual display of the bulk density data from the instrument at the sample site.

8. The method of claim 1, wherein:
the method further comprises collecting a soil sample of the sample site soil of the agricultural field; and
measuring percentage of carbon in the sample site soil comprises measuring the percentage of carbon in the soil sample.

9. The method of claim 8, wherein:
measuring the bulk density of the soil includes obtaining the bulk density data at the sample site;
the method further comprises transporting the soil sample to a location remote from the sample site;
measuring the percentage of carbon in the sample site soil includes measuring the percentage of carbon in the soil sample at the location remote from the sample site; and
determining the organic carbon content of the soil includes determining the organic carbon content at a location remote from the sample site.

10. The method of claim 9, wherein determining the organic carbon content of the soil includes determining the organic carbon content of the soil at the location that the percentage of carbon in the soil was measured.

11. The method of claim 1, wherein determining the organic carbon content includes:
determining the organic carbon content in the soil sample in a first set of units referenced to a first volume; and
multiplying the organic carbon content in the first set of units by a scaling factor to determine the organic carbon content in a second set of units referenced to a second volume greater than the first volume.

12. The method of claim 11, wherein multiplying by a scaling factor includes multiplying by a scaling factor to determine the organic carbon content in a second set of units suitable for an agricultural application.

13. The method of claim 12, wherein the second set of units comprises tons of carbon per acre.

14. The method of claim 1, comprising:
performing the steps of claim 1 for at least five sample sites per acre of a field including a plurality of contiguous acres to determine the organic carbon content at each of the at least five sample sites per acre; and
determining organic carbon content for the field based on the organic carbon content determined at the at least five sample sites per acre.

15. The method of claim 1, wherein:
the method further comprises measuring moisture content of the sample site soil to obtain moisture data; and
determining the organic carbon content further comprises determining the organic carbon content of the soil based on the moisture data.

16. The method of claim 15, wherein measuring the moisture content comprises measuring the moisture content of the sample site soil in situ.

17. The method of claim 16, wherein:
measuring the bulk density comprises measuring the bulk density of the sample site soil with a first instrument; and
measuring the moisture content comprises measuring the moisture content of the sample site soil with a second instrument different than the first instrument.

18. The method of claim 1 for determining the organic carbon content in agricultural field soil used to grow annual crops.

19. The method of claim 18 for determining the organic carbon content in agricultural field soil used to grow row crops.

20. A method in accordance with claim 1 for determining organic carbon content in agricultural field soil used to grow crops, comprising:
determining the organic carbon content in the agricultural field soil in accordance with the method of claim 1 before planting or substantial growth of the crops;
determining the organic carbon content in the agricultural field soil in accordance with the method of claim 1 after substantial growth or harvest of the crops; and
determining carbon sequestration in the agricultural field soil based on the organic carbon content before planting or substantial growth of the crops and the organic carbon content after substantial growth or harvest of the crops.

21. A method for determining organic carbon content in agricultural field soil used to grow crops, comprising:
receiving in situ-measured bulk density data of the soil of the agricultural field at a sample site, wherein the bulk density data was determined by measuring the bulk density of the sample site soil at the sample site in situ;
receiving a soil sample of the sample site soil of the agricultural field, wherein the soil sample was collected at the sample site;
measuring percentage of carbon in the soil sample to obtain percentage of carbon data; and
determining the organic carbon content of the soil based on the bulk density data and the percentage of carbon data.

22. The method of claim 21, wherein the bulk density data was measured by performing a nuclear density measurement at the sample site.

23. The method of claim 22, wherein the bulk density data was measured by operating a nuclear density gauge including a radioactive source.

24. The method of claim 23, wherein the bulk density data was measured by operating a nuclear density gauge exempt from licensing by applicable governmental laws or regulations.

25. The method of claim 24, wherein the bulk density data was measured in sample site soil undisturbed for the measurement.

26. The method of claim 25, wherein the bulk density data was measured by operating a density measurement instrument at the sample site.

27. The method of claim 26, wherein the density measurement instrument was operated by:
inserting a probe into the soil at the sample site; and
receiving a visual display of the bulk density data from the instrument at the sample site.

28. The method of claim 25, wherein:
the bulk density data was obtained at the sample site;
the soil sample was transported to a location remote from the sample site;
measuring the percentage of carbon in the soil sample includes measuring the percentage of carbon in the soil sample at the location remote from the sample site; and
determining the organic carbon content of the soil includes determining the organic carbon content at a location remote from the sample site.

29. The method of claim 25, wherein determining the organic carbon content of the soil includes determining the organic carbon content of the soil at the location that the percentage of carbon in the soil was measured.

30. The method of claim 26, wherein determining the organic carbon content includes:
determining the organic carbon content in the soil in a first set of units referenced to a first volume; and
multiplying the organic carbon content in the first set of units by a scaling factor to determine the organic carbon content in a second set of units referenced to a second volume greater than the first volume.

31. The method of claim 30, wherein multiplying by a scaling factor includes multiplying by a scaling factor to determine the organic carbon content in a second set of units suitable for an agricultural application.

32. The method of claim 31, wherein the second set of units comprises tons of carbon per acre.

33. The method of claim 32, comprising:
performing the steps of claim 21 for at least five sample sites per acre of a field including a plurality of contiguous acres to determine the organic carbon content at each of the at least five sample sites per acre; and
determining organic carbon content for the field based on the organic carbon content determined at the at least five sample sites per acre.

34. The method of claim 33, wherein:
the method further comprises receiving moisture data representative of moisture content of the sample site soil;
determining the organic carbon content further comprises determining the organic carbon content of the soil based on the moisture data.

35. The method of claim 34, wherein the moisture data was obtained by measuring the moisture content of the sample site soil at the sample site in situ.

36. The method of claim 35, wherein:
the bulk density data was obtained by measuring the bulk density of the sample site soil with a first instrument; and
the moisture data was obtained by measuring the moisture content of the sample site soil with a second instrument different than the first instrument.

37. The method of claim 26 for determining the organic carbon content in agricultural field soil used to grow annual crops.

38. The method of claim 37 for determining the organic carbon content in agricultural field soil used to grow row crops.

39. A method in accordance with claim 33 for determining organic carbon content in agricultural field soil used to grow crops, comprising:
determining the organic carbon content in the agricultural field soil in accordance with the method of claim 33 before planting or substantial growth of the crops;
determining the organic carbon content in the agricultural field soil in accordance with the method of claim 33 after substantial growth or harvest of the crops; and
determining carbon sequestration in the agricultural field soil based on the organic carbon content before planting or substantial growth of the crops and the organic carbon content after substantial growth or harvest of the crops.
